(19) United States
(12) Patent Application Publication
(10) Pub. No.: US 2005/0032151 A1
(43) Pub. Date: Feb. 10, 2005

(54) Methods of managing the transfer and use of data

(76) Inventors: Peter M. Eisenberg, Minneapolis, MN (US); Mitchell B. Grunes, Minneapolis, MN (US); Daniel K. McIntyre, St. Paul, MN (US); Diane E. Morel, Shoreview, MN (US); Paul A. Sevcik, Birchwood, MN (US)

Correspondence Address: 3M INNOVATIVE PROPERTIES COMPANY, PO BOX 33427, ST. PAUL, MN, 55133-3427, US

(21) Appl. No.: 10/478,583

(22) PCT Filed: Jun. 5, 2001
(86) PCT No.: PCT/US01/18157

(51) Int. Cl. C12P 35/02

(52) U.S. Cl. 435/51

(57) ABSTRACT

Certain improvements related to the transfer and use of information are disclosed, including a the transfer of information from an existing database to a database used in conjunction with an RFID device of the type that may be used to interrogate RFID tags associated with items associated with entries in the existing database.

### TECHNICAL FIELD

The present invention relates to improvements in methods of managing the transfer and use of data, and in one embodiment the transfer and subsequent use of data from an existing database having an arbitrary data management system to a selected data management system.

### BACKGROUND OF THE INVENTION

Many facilities have computers that include databases with entries describing multiple items. One example is a library, which typically has a computer with a database including entries for each library book, magazine, or other material possessed by the library. The database may be provided by a vendor, such as a library automation vendor. Those databases enable a library to access data related to one, a group of, or all of the materials in the library, as needed. For example, if a patron requests a particular book, the database can provide information regarding the circulation status of the book, such as the most recent date on which it was checked out, and other related information. These types of databases are common in other fields also, including asset tracking and management generally.

In some fields, there are a variety of databases that use file formats that are not inter-compatible, and thus retrieving information from the database of one system for use with other systems can be problematic. For example, a particular university library may have a database listing its materials that is different from the corresponding database that a particular public library maintains, which in turn may be different from the database that a particular junior high school library maintains. It therefore becomes difficult for equipment, software, service or other suppliers to interact effectively with each of these different databases without customizing those databases. Because manual customization, or entry or re-entry of the contents of an entire database can be an impossibly large task, there is a need for improvements in the transfer and use of information between different databases. That is the subject of the present invention.

### SUMMARY OF THE INVENTION

The present invention includes a variety of features described herein, including a method of transferring and using information, comprising the steps of obtaining information related to a plurality of items from an existing database; reformatting the information in a desired manner to facilitate the use of the information by an RFID reader; exporting the information to a database stored on a data storage device; and using the information on the data storage device with an RFID reader in conjunction with the interrogation of RFID tags associated with the items. A combination of software for reformatting information obtained from an existing database having an arbitrary data management system into reformatted information stored in a database for use by an RFID reader, the databases comprising entries related to items of interest; and an RFID reader that interrogates RFID tags associated with items and transfers information related to the interrogated RFID-tagged item from the RFID reader to the database, from the database to the RFID reader, or both, is also disclosed.

The data transfer and management system of the present invention may be used in conjunction with devices such as a portable RFID reader, self-service terminals and staff workstations for processing tagged items, conversion stations, and other item processing devices. The system may be used not only in connection with RFID-tagged items, but also items that are associated with other item identifiers, such as barcodes, characters, handwritten indicia, and other types of identification.

The present invention, which typically uses lists or files created from an existing database, has several advantages over systems that attempt to provide direct access to an existing database. First, direct access systems require detailed knowledge of the structure of the existing database and how to create a connection to that database. Because the structures may differ based on the database, as described above, direct connections may be difficult to obtain. Second, direct access is relatively slow compared to file access (as used herein), because the existing database is generally on a different computer and requested data has to be separated from unrequested data within that database. Extraction into a file provides faster access than by direct query. Third, some existing databases may not support standard access, such as SQL access, but essentially all existing databases should be able to provide some kind of reporting features for generating list files.

The items that are the subject of the data may be assets of any kind, including library materials, criminal evidence, documents or files, containers, pallets, boxes, retail goods, rental items, video tapes, or laboratory samples.

These and other aspects of the present invention are described in greater detail below.

### DETAILED DESCRIPTION OF THE INVENTION

The present invention is described in some instances with reference to the management of data in the context of a library, and specifically in interacting with existing databases of the type described above. However, the usefulness of the present invention is not limited to the management of data in libraries, as will be evident from the following disclosure. The data transfer and management methods and systems of the present invention enable a user to extract data from an existing database, transfer them into a new database, reformat those data, and then use the reformatted data in a desired manner. Those and other aspects of the present invention will be described in detail below, and are also described in a publication entitled 3M Digital Data Manager Model 747 User Guide, a copy of which was submitted in an Information Disclosure Statement accompanying the present application, and the entire contents of which is incorporated by reference herein (hereinafter the “Data Manager User Guide”). Where additional information may be useful to supplement an understanding of the present invention, reference is made to the subject matter incorporated from the Data Manager User Guide. Although the following description is provided largely in the context of transferring information from an existing arbitrary database to a new database, the reverse processes can also be implemented to reformat information in a manner suitable for transfer to an existing database.

Transfer and Use of Information

In one aspect of the invention, a method is provided for obtaining data from an existing database through a user interface (such as that provided on a personal computer), downloading the data to a new database, optimizing or reformatting those data in a way that enables a radio frequency identification (“RFID”) device to use the data, downloading the optimized or reformatted data to a non-volatile data storage medium, loading the data storage medium into an RFID device (preferably a portable, handheld RFID reader, though other non-handheld devices of the type mentioned below are also suitable), and then using the RFID device in conjunction with the data to obtain real-time feedback from the RFID device as to items having RFID tags that are interrogated by the RFID device. The RFID device may be an RFID reader (or interrogator) of the type referred to in the Data Manager User Guide as the Digital Library Assistant, or “DLA,” which is available from Minnesota Mining and Manufacturing Company of St. Paul, Minn. (“3M”). Additional information related to RFID devices of this type is available in, for example, U.S. Pat. No. 6,232,870 (Garber et al.), the contents of which is incorporated by reference herein, and from other manufacturers of RFID devices including Texas Instruments of Dallas, Tex.

A. Preparing Database Entries for Export

Data from a database may be prepared for eventual use by an RFID device, for example, in the following manner. The existing database is typically created and maintained on a personal computer or on a server, and may include thousands or millions of entries related to items of interest. First, folders may be created (in a Windows™ operating system environment, for example) that store files containing data extracted from the database. Those folders may be, for example, ones that contain files listing items in a specific order (such as the order in which books or files are supposed to be positioned on a shelf, or the order in which other assets are supposed to be positioned within a warehouse, or simply in order of their serial numbers), or ones that contain files with lists of items for which the user wishes to search (such as items thought to be missing, items that can be retrieved and discarded, items that a person wishes to retrieve for herself or another user, or other such things). These folders can reside on a hard drive, on a network drive, on a removable data storage medium, or on any type or combination of data storage media Folders may be useful for storing multiple files that are related to each other, thereby linking files that describe common items, such as items located adjacent to each other in within a storage area, or for other reasons. One reason that a user might choose to create multiple files that are linked in a folder is, for example, if a list file containing the records selected for use is quite large, then it may be useful to split that file into two or more files that can be stored within the same folder. Thus the ordered list folder may contain a first file having information identifying items that are supposed to be located on the first 100 locations within the storage area, a second file having information identifying items that are supposed to be located on the second 100 locations within the storage area, and so on. To prepare the data placed into the folders for transfer, an export location is designated. Typically the export location is a removable data storage device, such as a flash memory card, floppy disc, or the like. The export location may also be a hard drive or a network drive. There could also be multiple export locations for the same information.

In the following example, the data stored in the files extracted from the existing database includes item identifiers (such as barcodes, characters, hand-written identifiers, or the like) associated with items. The item identifiers may be reformatted in a manner that makes them easy to read in a printed report, such as adding spaces and other formatting characters, though the added information may not form part of the actual item identifier. Although it is preferred that the data manager not select the data directly from the existing database, but rather obtain data from a list of data selected by a user, the data manager could directly select the data from the existing database and place it into files as described above, or could select the data from the existing database based on a specified format of the existing data, or a user could create an intermediate database or text file with data in a specified format that the data manager can extract. To validate the item identifier information from the existing database, a valid length parameter is assigned. For example, for barcodes the minimum barcode length may be 14 characters, and the maximum barcode length may be 16 characters, though other valid length parameters may be selected depending on the type and format of the item identifier(s) in the existing database. The length parameters could also be the same, so that only item identifiers of a specific length would be accepted. Valid characters are also designated, so that the data transfer system can recognize those characters and ignore all others. Examples of valid characters may include numeric digits (0 through 9), lower case letters (a through z), upper case letters (A through Z), or additional, user designated characters (such as one or more of the symbols !@#$% \hat{ }&*( )_+?><”:’;}{][=−\|). The user may customize the data format by setting values for format configuration parameters. Data that do not meet the format configuration parameters can be identified, which is advantageous because it enables the data manager to inform a user, or create a log, concerning invalid item identifiers such as barcodes so that the user can correct these identifiers in the existing database.

In some instances, an item identifier provided on an item does not match the corresponding item identifier from the list file, and thus from the existing database record. When this occurs, it is necessary to alter the identifier of the item so that it corresponds to the identifier stored in the existing database. This is done using filters, which in the case of filters for barcodes are simply referred to as barcode filters. A filter such as a barcode filter consists of a group of instructions that can identify a specific barcode data format, and then change that format so that it matches the existing database data format. The filter instructions include conditions and actions. Conditions are requirements that must be met before the filter can be applied. For example, the required item identifier length is a condition. If an item identifier meets all of the conditions in the filter instructions, then the filter will apply all the actions contained in the filter instructions. For example, an action may be to find a specific character string in an item identifier such as a barcode and replace it with a desired character string. The data manager system provides a way to filter the item identifier so that the identifier read from the item will match the item identifier read from the list file. Another condition may be the existence of a specified string of characters in the item identifier. Other actions include adding specified characters, padding the item identifier on the leading or trailing end of the identifier, or both, until it reaches a specified length, adding a check digit computed using a selected algorithm, removing specified characters, or replacing specified characters. Additional information related to filtering, such as barcode filtering, is provided in the Data Manager User Guide incorporated by reference above.

The format in which the user's data exists, whether the data is in the user's existing database or in data files extracted from the existing database by the user, must be designated in order for the data manager to extract the data from the existing database or from the extracted data files. For example, the data format of files extracted from an existing database may be selected by the user from among a number of proposed formats provided by the data manager software package, or may be customized by the user. For example, the user may specify that each item record in the existing database includes multiple lines of text, and that the records are separated by blank lines. Another format may include ones in which each record is provided on a single line, with a particular user-defined delimiter, such as a tab character, separating fields within the record. Yet another format may include ones in which each record is on a single line, and the field boundaries are defined by a fixed width, or number of characters. As a specific example of a data format, the user may specify that the first 12 lines of the database should be skipped, then that the item identifier starts at, for example, the 6^{th} character position on the second line of each item record and extends for 12 characters, and may also specify the locations of the primary and secondary information in the records. User-defined formats may be named, saved, edited, reused, specified as a potential default format for future use, or more than one of the foregoing. Concurrent with identifying the format of the files extracted from the existing database, the user may select the data to be displayed on an RFID device that is useful for an operator. For example, certain information from each database record may be designated for display on an RFID device as a primary information field, and other information from that database record may be designated for display as a secondary information field. Any number of information fields may be provided, and the corresponding information displayed for the user. For example, the user may wish to display on the first line of a display the name and/or title of an object or a portion of the name and/or title of an object, and to display on the second line of a display an identification number, call number, serial number and/or the equivalent or a portion of an identification number, call number, serial number and/or the equivalent. Those fields would be indicated as the primary and secondary information fields, respectively. Additional fields may also be designated relative to each database record, perhaps related to information about borrowing activity for each item, or the date of publication, or in the case of certain items the date the item was made or shipped, and that information may also be displayed for a user.

Another aspect of the data manager system of the present invention is the ability of the system to handle duplicate items, duplicate database records on a single list file, duplicate database records on different list files, or any combination of these issues. The data manager system can address duplicate items by comparing a primary or secondary information field (which might include an identification number, call number, or the like as described above), and then treating as duplicate entries those that match one or both of the primary and secondary information fields. Thus in general terms, the data manager identifies as a single item or type of item things that have differing item identifiers by, for example, comparing one or more information fields related to each entry to determine whether they are identical. For example, if a facility has multiple duplicate items, then the associated database may contain a corresponding number of substantially identical entries. This can occur in a library where, for example, 10 duplicate copies of a library book may be available for patrons, or in a warehouse where 10 identical products are all designated with the same identifying information. In this instance, then it may not matter to a user whether the duplicate items are in any particular order relative to each other, so long as they are all located together. When a ordered list of items (such as a list of items in the order they should be stored in a storage area) is prepared, the data manager assigns the same storage area location to each identical item. That way, none is considered by, for example, an RFID reader to be out of position so long as it is located with other like items. Another manner of addressing the same situation is to designate a range of acceptable locations for each of the multiple items, and to instruct the data manager system that if the item is found somewhere within that range of positions, it should be considered to be in the correct position. Thus where there are ten identical items, and each can be located anywhere between shelf position 3395 and 3404, the data manager can be instructed to consider that to be a proper location.

The data manager may also address a situation where duplicate database records are on a single list file or duplicate database records are on different list files. If multiple database records exist in the same ordered list, or on more than one ordered list for the same storage area, this can assumed to be an error because an item cannot physically be in two or more locations at the same time. The data manager software can detect and report this, thus allowing the user to correct the mistake in the existing database.

Transferring large files extracted from an existing database to a new database using the data manager system and methods of the present invention can take a substantial period of time, particularly when a large number of database records are transferred. This time can be wasted if the database records are not properly transferred and formatted for the new database. A “preview file export” feature may be provided, so that prior to transferring 25,000 database records, for example, the first 1000 records can be displayed for the user to review before proceeding to export all the records. In the case of database records selected by the user as described above and stored in a file such as an ordered list for use by the data manager system, the preview file export feature may include fields including the storage area location (in numerical format, for example), the item identifier, call number or the like, the primary information field, the secondary information field, and any other fields desired by the user. Once the user has reviewed the files that have been previewed, the user can proceed to export the entire set of database records, or a subset, by initiating the appropriate commands.

B. Exporting Data

One or more data lists can be selected for export to a data storage device, such as a hard drive or, preferably, the removable data storage medium of the type referenced above. That data storage device may be non-volatile, an example of which is a compact-flash memory card, which is a solid-state data storage medium that can be inserted into and withdrawn from a compact flash drive or port. Additional information related to removable data storage media is provided in copending PCT Application No. PCT/US01/07979, filed Mar. 13, 2001 and entitled “Radio Frequency Identification Reader with Removable Media,” the contents of which is incorporated by reference herein.

In another embodiment, the data may be exported to a data storage device that is docked or otherwise connected (for example by a hardwired connection to a piece of hardware, or by a tethered connection to a piece of portable hardware) to the processor that exports the data, or could be transferred by wireless connection, all in a manner known in the art.

At the conclusion of the export, a summary log can be displayed for or made available to a user. The log may include a description of the files that were transferred, the number of records that were transferred, the elapsed time for the transfer, the number of errors encountered, error messages and warnings, and similar information. If errors are noted, then access may be provided to a detailed export or error log, which can describe the reason that an error was detected. For example, a database record may have been lacking information in a required field (such as the item identifier, call number, title, or the like), or have an invalid character, or the like. Or the extracted file(s) may contain multiple entries setting forth different positions that a single item is located within the library. This information is useful because the user can then correct the existing database, so that the integrity of the existing database is improved. Either the new or the existing database or both could then be searched to locate all entries with a particular type of error, such as the absence of an item identifier, or the absence of a name or title. When the export is complete, the new database containing the exported files may be transferred electronically, or a removable data storage medium may be withdrawn from a drive and inserted into another drive or port, for example.

The information formatted and exported from the data manager as described above can then be stored on the user's same computer, on a portable RFID device, or, preferably, on the removable data storage device through an appropriate docking station, all as described above. When that memory device is next inserted into the RFID reader, the reader is provided with access to data stored in a uniform manner, from which other operations of the reader can draw.

C. Importing Data Collected from Interrogated RFID Tags to an Existing Database

In another embodiment of the present invention, after data has been collected by an RFID reader by interrogation of RFID tags associated with items of interest, the data may be uploaded to an existing database. This may be done by a process similar to reversing the process described above for extracting data from an existing database, transferring the data to a new database and then reformatting those data so that the reformatted data can be used in a desired manner. That is, the collected data is uploaded from the database in which it is stored after collection by the RFID reader to the system on which the data manager is resident. The data manager reformats the collected data by the data transfer and management methods of the present invention to the format of the existing database using database format designations provided by the user to the data manager. The reformatted data is then uploaded to the user's existing database. If the existing database is not accessible to the data manager, the reformatted collected data is transferred to a storage location accessible to the data manager for later uploading to the user's database. For example, the reformatted data may be stored on a removable data storage medium that is in communication with a portable RFID reader, and that data storage medium can be used to import or upload the data to the existing database at any appropriate time.

In another embodiment, an RFID reader may be used to collect data without using information supplied by the user. In order to reformat the collected data to a format compatible with the existing database, the user first designates to the data manager the format in which the data exists in the existing database (to which the user intends to upload the collected information), or designates the format in which the existing database expects to receive the data. The reformatting of the collected data by the data manager and uploading of the reformatted collected data to the existing database occurs as described above.

In some cases the existing database management system may not be capable of directly uploading the reformatted collected data from data files. In such cases, the user may upload the reformatted collected data to the existing database via a software keyboard wedge. This is a software application that can run on a computer that is operating the existing database client application or a terminal emulator connected to the user's database. The purpose of the software keyboard wedge application is to read data from a file on the computer and translate the data in the file into keyboard input which can be accepted by another program running on the computer, in this case the database client application or terminal emulator. To use the software keyboard wedge, the user would first set the database client application or terminal emulator to accept input, for example identifiers. Then the software keyboard wedge would be activated and configured to provide data from the data file into the application in focus or another application. In this case the target is the database client application or terminal emulator. The operation will appear to the database as if the item information had been entered manually at the keyboard by the user.

The data transfer and management system of the present invention may also be used in conjunction with devices other than a portable RFID reader. For example, self-service terminals and staff workstations for processing tagged items, such as those sold by the assignee of the present invention under the designation “Digital SelfCheck™ System” and “Digital Staff Workstation,” may also use data transferred to either or both of them in the manner described herein. Stations for converting barcoded items to RFID-tagged items, such as those sold by the assignee of the present invention under the designation “Digital Conversion Station” may also be used with the system of the present invention. Optical character recognition systems, manual entry systems, and list-based conversion systems may also be used. In that manner, items being processed by hardware other than a portable reader could also be checked against inventory or other lists, and handled appropriately.

Although RFID and non-RFID systems are not in general interchangeable, the present invention lends itself to non-RFID systems also. That is, barcode, optical character recognition (OCR), handwriting, or other readers and systems could be effectively substituted for RFID-based systems of the type described herein. In that manner, information stored in a database could be designated, reformatted, exported, and used by a barcode or OCR-based system in the same manner as with an RFID system, despite the obvious differences between RFID-based and optically-based identification systems, because the specific type of interrogation system is less important than the data transfer and management systems of the present invention. This would enable someone using a barcode or OCR scanner to search for particular items among a group of items, for example. Barcode and OCR scanners and systems are well known in the art.

The items described herein may be library materials, but may also be files (of the type commonly used to store paper, as opposed to electronic files), patient or client records, assets, retail and consumer goods, pallets or containers, or other similar items.

1. A method of transferring and using information, comprising the steps of:
(a) obtaining information related to a plurality of items from an existing database;
(b) reformatting the information in a desired manner to facilitate the use of the information by an RFID reader;
(c) exporting the information to a database stored on a data storage device; and
(d) using the information on the data storage device with an RFID reader in conjunction with the interrogation of RFID tags associated with the items.

2. The method of claim 1, wherein the existing database includes information correlating the items to item identifiers.

3. The method of claim 2, wherein the item identifiers comprise barcodes.

4. The method of claim 2, wherein the item identifiers comprise at least one of characters and handwriting.

5. The method of claim 1, wherein the information exported to the data storage device comprises at least one ordered list of items.

6. The method of claim 5, wherein the ordered list is a list of items in an order that the items are to be located in a storage area.

7. The method of claim 1, wherein the information exported to the data storage device comprises at least one search list of items.

8. The method of claim 1, wherein the data storage device is a removable non-volatile data storage device.

9. The method of claim 8, wherein the removable non-volatile data storage device is a solid-state device.

10. The method of claim 9, wherein the removable non-volatile solid-state data storage device is a compact flash memory card.

11. The method of claim 1, wherein the information exported to the data storage device comprises more than one file, each file including at least one database record.

12. The method of claim 11, wherein the method further comprises the step of associating at least two files that include information describing database records related to consecutive items in an ordered list.

13. The method of claim 11, wherein the method further comprises the step of determining that two files do not include information describing database records related to consecutive items.

14. The method of claim 1, wherein the information is reformatted by selecting from each record in the existing database information to be provided in a primary information field and information to be provided in a secondary information field on the database on the data storage device.

15. The method of claim 14, wherein at least one of the information fields comprises information from a record related to a single type of information.

16. The method of claim 14, wherein at least one of the information fields comprises information from a record related to more than one type of information.

17. The method of claim 14, wherein at least one of the information fields comprises information from a record in the existing database that represents only a portion of the information contained in that record.

18. The method of claim 14, wherein the information selected for the primary and secondary information fields is selected from the group consisting of the name or title of the item, the identification number of the item, or the call number of the item.

19. The method of claim 14, wherein the method further comprises the step of displaying information obtained from the primary information field and information obtained from the secondary information field on the RFID device for observation by a user.

20. The method of claim 1, wherein the step of reformatting the data comprises identifying multiple records in the existing database that relate to equivalent items.

21. The method of claim 20, wherein the method further comprises the step of providing only one entry on the database stored on the data storage device relative to that item.

22. The method of claim 20, wherein the method further comprises the step of comparing multiple entries from the existing database to determine whether the multiple entries relate to equivalent items using at least one of a primary information field and a secondary information field.

23. (Cancelled)

24. The method of claim 23, wherein the method further comprises the step of assigning the same storage area location to each identical item.

25. The method of claim 23, wherein the method further comprises the step of assigning a range of storage area locations to each identical item, so that each such item located within the range by the RFID device is considered by the device to be in the proper location.

26. The method of claim 1, wherein the method further comprises the step of previewing the format of at least one entry for the database on the data storage device prior to step (c).

27. The method of claim 26, wherein the entry includes information selected from the group consisting of an item identifier, a primary information field, a secondary information field, and a barcode.

28. The method of claim 1, wherein the method further comprises the step of providing a summary log related to the exportation of information.

29. The method of claim 28, wherein the summary log comprises information selected from a group consisting of a description of the files that were exported, the number of entries that were exported, the elapsed time for the transfer, and the number of errors encountered during the export.

30. The method of claim 28, wherein the method further comprises providing a detailed error log that provides information related to errors detected in the exported information.

31. The method of claim 1, wherein the items are library materials.

32. The method of claim 1, wherein the items are files.

33. The method of claim 1, wherein the items are pieces of evidence.

34. The method of claim 1, wherein the items are pallets or containers.

35. Software comprising instructions for carrying out the method of claim 1.

36. In combination:
(a) a data transfer and management system software for reformatting information obtained from an existing database having an arbitrary data management system into reformatted information stored in a database for use by an RFID reader and for reformatting information obtained from a database containing information for use by an RFID reader into reformatted information stored in an existing database having an arbitrary data management system, the databases comprising entries related to items of interest; and
(b) an RFID reader that interrogates RFID tags associated with items and stores information related to the interrogated RFID-tagged item in a database used by the RFID reader.

37. The combination of claim 36, wherein the RFID reader is a handheld RFID reader.

38. The combination of claim 36, wherein the RFID reader is a component of a workstation for processing items selected by users.

39. The combination of claim 38, wherein the workstation is a workstation adapted for use by a library employee.

40. The combination of claim 38, wherein the workstation is a self-service station adapted for use by the user who selected the items.

41. The combination of claim 38, wherein the workstation is a conversion station for converting non-RFID-tagged items to RFID-tagged items.

42. The combination of claim 41, wherein the non-RFID-tagged items are barcoded items.

43. The combination of claim 41, wherein the non-RFID-tagged items are identified by characters.

44. The combination of claim 41, wherein the non-RFID-tagged items are items regarding which a user enters information describing the items into the workstation.
